(19) United States
(12) Patent Application Publication
(10) Pub. No.: US 2026/0346812 A1
(43) Pub. Date: Nov. 5, 2026

(54) VEHICLE WITH INTEGRATED TACHOGRAPH

(71) Applicant: Ford Global Technologies, LLC, Dearborn, MI (US)

(72) Inventors: Stuart C. Salter, White Lake, MI (US); Jack Lawrence Jackson Van hoecke, Austin, TX (US); David Celinske, Wolverine Lake, MI (US); Aiden James McDonagh, Rayleigh (GB)

(73) Assignee: Ford Global Technologies, LLC, Dearborn, MI (US)

(21) Appl. No.: 19/196,464

(22) Filed: May 1, 2025

(51) Int. Cl. B60R 25/22; A61B 5/18; B60R 16/033; B60R 25/04; B60R 25/06; B60R 25/102; B60R 25/30; B60R 25/31; G06K 13/02; G06V 20/59; G06V 40/10; G07C 5/04; G07C 5/06

(52) U.S. Cl. CPC B60R 25/22; B60R 16/033; B60R 25/04; B60R 25/066; B60R 25/102; B60R 25/305; B60R 25/31; G06K 13/02; G06V 20/597; G06V 40/10; G07C 5/04; G07C 5/06; A61B 5/18

(57) ABSTRACT

A vehicle including a tachograph, a sensor unit and a processor is disclosed. The sensor unit may capture inputs associated with a vehicle interior portion and vehicle's surroundings. The processor may determine that a tachograph card is connected to the tachograph. Responsive to such determination, the processor may authenticate a driver based on inputs obtained from the sensor unit and driver information obtained from the tachograph card. The processor may perform a predefined action when the driver is not authenticated.

### FIELD

The present disclosure relates to vehicles, and more specifically, to a vehicle with an integrated tachograph.

### BACKGROUND

A tachograph is a device that is used in commercial vehicles to record driver's activities such as the driver's drive time, rest time, total distance traveled, and/or the like. Each driver typically has a tachograph card that the driver inserts into the tachograph, which then records and registers the driver's activity on the card. Recording such driver's activities is often desirable for commercial vehicles. This recording also facilitates fleet managers' ability to optimize the driver's drive and rest times and promote healthy working conditions for the drivers.

### BRIEF DESCRIPTION OF THE DRAWINGS

The detailed description is set forth with reference to the accompanying drawings.

The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale. Throughout this disclosure, depending on the context, singular and plural terminology may be used interchangeably.

FIG. 1 depicts an example vehicle with an integrated tachograph in accordance with the present disclosure.

FIG. 2 depicts a block diagram of a system to operate a vehicle with an integrated tachograph in accordance with the present disclosure.

FIG. 3 depicts an example view of a driver leaving a vehicle with a tachograph card inserted in a tachograph in accordance with the present disclosure.

FIG. 4 depicts an example view of a parked vehicle in accordance with the present disclosure.

FIG. 5 depicts an example view of a vehicle's Human-Machine Interface (HMI) in accordance with the present disclosure.

FIG. 6 depicts a flow diagram of an example method to operate a vehicle with an integrated tachograph in accordance with the present disclosure.

### DETAILED DESCRIPTION

### Overview

The present disclosure describes a vehicle with an integrated tachograph. By integrating the tachograph into the vehicle, the tachograph is able to use the vehicle's existing sensors, which significantly enhances the tachograph's performance and enriches the driver's experience of operating the tachograph. Specifically, by integrating the tachograph, the vehicle is able to provide a plurality of additional features or benefits to the driver, fleet managers, and/or the like, which may not be possible in conventional systems where the tachograph is attached to the vehicle as an aftermarket accessory. Examples of such additional features are briefly described below.

In one exemplary aspect, the vehicle may use the vehicle's existing interior camera(s) to authenticate the driver, when the driver inserts a tachograph card (“card”) associated with the driver into the tachograph and attempts to drive the vehicle. The vehicle may output an alert notification to a user device/computing device/server associated with the fleet manager or disable the driver from driving the vehicle when the driver is not authenticated, thereby preventing unauthorized use of the card and/or the vehicle.

In another exemplary aspect, the vehicle may enable the card to act as a vehicle key, thereby enabling the driver to start/drive/open the vehicle by using the card. The vehicle may further enable the driver to use the vehicle's microphone or the vehicle's sensor unit to provide inputs to the tachograph or record activities via audible or gesture commands.

The vehicle may further output an alert notification when the driver exits the vehicle while leaving the card inserted in the tachograph. In another exemplary aspect, the vehicle may output another alert notification when the vehicle determines that the driver's drive time is close to exceeding the allocated drive time left on the card. This alert notification may remind the driver about taking rest, and may hence promote healthy driving habits.

The vehicle may further use the input obtained from the vehicle's sensor unit to check if the driver's respiration or heartbeat has increased and may output a recommendation for the driver to take a rest break or increase the rest time responsive to such determination. The vehicle may further use the vehicle's Human-Machine Interface (HMI, which may have a considerably larger display screen than the tachograph's screen) to display driver and/or trip related information, which may greatly enhance the driver's experience of operating the vehicle / tachograph. For example, the vehicle may cause the HMI to display information associated with driver history stored on the card, upcoming week's activities with allowed driving time, countries visited and tolls paid historically, paper left in the tachograph printer, last 15 or 28 days of driving record, and/or the like.

The present disclosure discloses a vehicle that provides various additional features and benefits to the driver (and/or the fleet manager) by integrating the tachograph into the vehicle, which is not possible in conventional systems in which the tachograph is added as an aftermarket accessory to the vehicle. The integrated tachograph may use the existing vehicle sensors, which significantly enhance the tachograph's performance and the driver's experience of using the tachograph. This further enables the fleet manager to remotely access the driver's records, thereby eliminating the need for the fleet manager to go to the vehicle and obtain a printout from the tachograph.

These and other advantages of the present disclosure are provided in detail herein.

### Illustrative Embodiments

The disclosure will be described more fully hereinafter with reference to the accompanying drawings, in which example embodiments of the disclosure are shown, and not intended to be limiting.

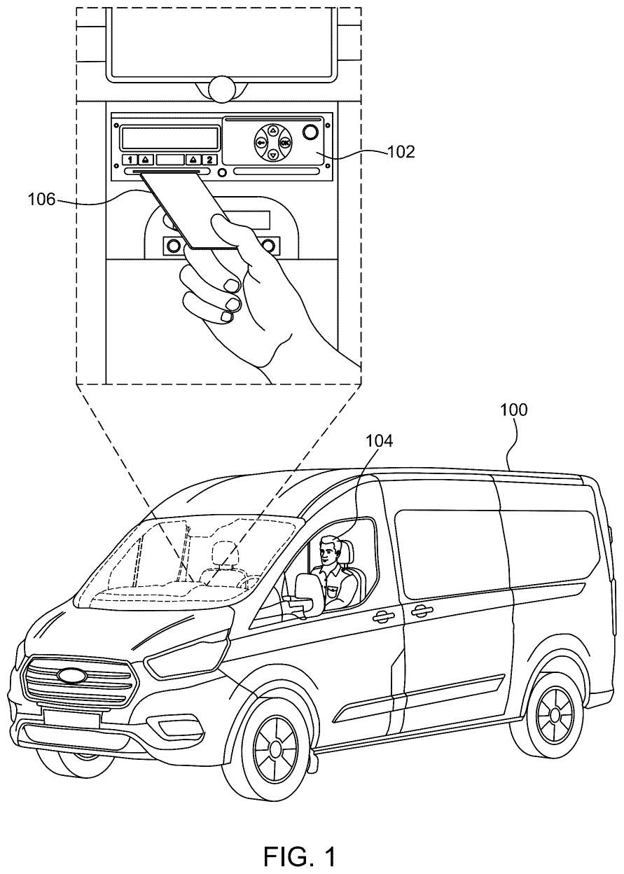

FIG. 1 depicts an example vehicle 100 with an integrated tachograph 102 in accordance with the present disclosure. The vehicle 100 may take the form of any passenger or commercial vehicle such as a car, a work vehicle, a crossover vehicle, a truck, a van, a minivan, a taxi, a bus, etc. The vehicle 100 may be a manually driven vehicle or may be configured to operate in a partially/fully autonomous mode. Further, the vehicle 100 may include any powertrain, such as a gasoline engine, one or more electrically actuated motor(s), or a hybrid system.

The tachograph 102 may include a plurality of components including, but not limited to, a tachograph head unit (as shown in FIG. 1) that may be located on the vehicle's dashboard, a tachograph card reader, a printer, one or more buttons to receive driver's inputs, and/or the like. It is known that a tachograph (e.g., the tachograph 102) is a device used in commercial vehicles (e.g., trucks, buses, etc.) to record driving and rest times associated with a driver (e.g., a driver 104 depicted in FIG. 1), vehicle's speed, distance traveled, etc. The tachograph 102 facilitates the driver 104 to drive the vehicle 100 as per the best practices defined by the authorities and take the recommended amount of rest during a trip. The tachograph 102 also facilitates the authorities and/or the vehicle's fleet manager to check if the driver 104 is driving the vehicle 100 as per the defined best practices and adhering to healthy driving and rest behavior.

In some aspects, the driver 104 may be allocated a tachograph card 106 that may be a smart card with digital tachographs. Whenever the driver 104 desires to drive the vehicle 100, the driver 104 may be required to insert the tachograph card 106 into the tachograph card reader of the tachograph 102 (as shown in FIG. 1). The tachograph card 106 stores driver information including, but not limited to, driver's name and identity, driver's image, historical driver's drive time and rest time, historical driving speed, distance traveled, locations traveled, and/or the like. Most of the information stored on the tachograph card 106 is determined by the tachograph 102. For example, when the driver 104 is driving the vehicle 100, the tachograph 102 determines the driver's drive time, rest time, driving speed, distance traveled, locations traveled, and/or the like, and stores this information on the tachograph card 106 as the driver's information or “record”.

Such stored information may be accessed by the authorities and/or the vehicle's fleet manager via the tachograph card 106, to check the activities and/or performance associated with the driver 104.

In conventional systems, a tachograph is an external unit that is attached to a vehicle. In such systems, the tachograph includes its own antenna and motion sensors, which detects the vehicle's driving speed, location, rest or off time, and/or the like. While such conventional systems do capture the driver's activities to some extent, the conventional systems are not able to optimally use the vehicle's existing sensors, components, etc. Use of vehicle's existing sensors, in conjunction with a tachograph, can greatly enhance the tachograph's performance and provide additional features to the driver 104, the fleet managers, and/or the like, which may enrich the experience of operating the tachograph.

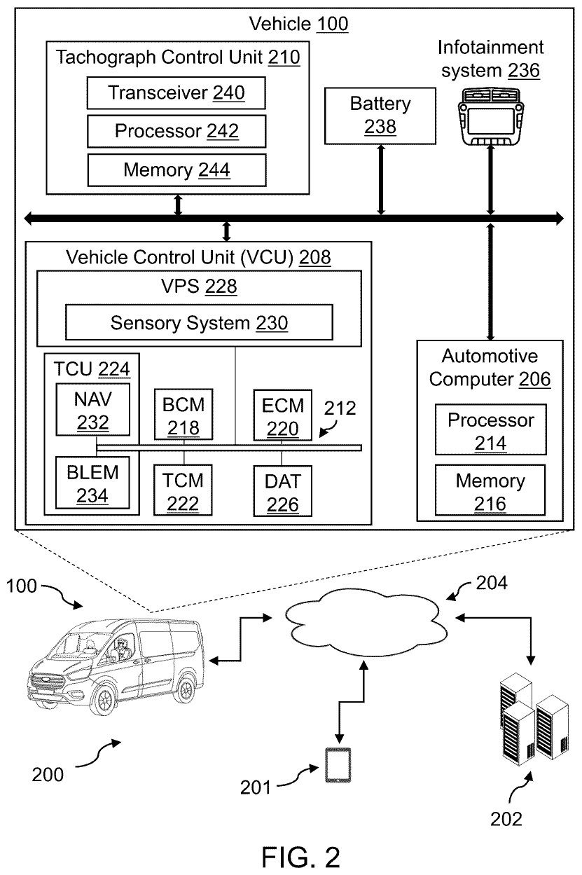

In light of this, the present disclosure proposes the vehicle 100 that has the tachograph 102 integrated into it. Specifically, the tachograph 102 is integrated into the vehicle 100 at the vehicle's manufacturing or assembly stage, thereby removing the need to externally attach the tachograph 102 to the vehicle 100 after purchase. Further, in an exemplary aspect, since the tachograph 102 is integrated with the vehicle 100, the tachograph 102 uses the vehicle's existing V2X antenna (or vehicle-to-everything antenna) and motion sensors to record the driver's activities. Furthermore, due to the integration of the tachograph 102 with the vehicle 100, the vehicle 100 is able to provide a plurality of additional features to the driver 104, the fleet managers, and/or the like, which may not be possible in conventional systems. Examples of such additional features are briefly described below and described in detail later in conjunction with FIG. 2.

In one exemplary aspect, the vehicle 100 may use the vehicle's existing interior camera to authenticate the driver 104 when the driver 104 inserts the card 106 into the tachograph 102 and attempts to drive the vehicle 100. In this case, as an example, the vehicle 100 may first obtain the driver's image that is recorded on the card 106. The vehicle 100 may then compare the obtained driver's image with the real-time driver's image that is captured by the vehicle's interior camera (e.g., when the driver 104 enters the vehicle 100 and sits on the driver's sitting area). The vehicle 100 may enable the driver 104 to use the vehicle 100 when the two images match (meaning the driver 104 is authenticated), and may perform one or more predefined actions when the two images do not match (meaning the driver 104 is not authenticated). For example, in the latter case, the vehicle 100 may output an alert notification to a user device/computing device/server associated with the fleet manager, informing the fleet manager that an unauthenticated driver is attempting to drive the vehicle 100. As another example, the vehicle 100 may disable the driver 104 from driving the vehicle 100 when the driver 104 is not authenticated (e.g., not enable the vehicle 100 to move out of park in this case).

In another exemplary aspect, the vehicle 100 may enable the card 106 to act as a vehicle key, thereby enabling the driver 104 to start/drive the vehicle 100 by inserting the card 106 into the tachograph 102. This may ensure that the driver 104 does not forget to insert the card 106 into the tachograph 102 before driving the vehicle 100. The vehicle 100 may further enable the driver 104 to move the vehicle 100 out of park only when the driver 104 inserts the card 106 into the tachograph 102. In some aspects, the vehicle 100 may determine that the card 106 is inserted into the tachograph 102 based on signals obtained from the tachograph 102. As an example, the tachograph 102 may generate a trigger signal when the card 106 is inserted into it. A vehicle processor (shown as processor 242 in FIG. 2) may receive this trigger signal and determine that the card 106 is inserted into the tachograph 102 responsive to receiving the trigger signal. The vehicle 100 may then enable the driver 104 to move the vehicle 100 out of park mode.

In another exemplary aspect, the vehicle 100 may enable the driver 104 to use the vehicle's microphone or the vehicle's sensor unit (shown as vehicle sensory system 230 in FIG. 2) to provide inputs to the tachograph 102 or record activities via audible or gesture commands. For example, the driver 104 may provide inputs associated with whether the driver 104 is performing some other work different from driving (e.g., loading, unloading, paperwork, etc.) or taking rests, the driver's available status, print requests, and/or the like, to the tachograph 102 via audible or gesture commands that are captured by the vehicle's sensor unit/microphone. In this case, the vehicle's sensor unit/microphone may captured the driver's audible or gesture commands and transmit them to the tachograph 102 (which can store these driver's inputs onto the card 106 as driver information/records).

In yet another exemplary aspect, the vehicle 100 may use the inputs obtained from the vehicle's sensor unit to check/determination if the driver 104 has exited the vehicle 100 (or an immediate area of the vehicle 100) and left the card 106 inserted in the tachograph 102. In this case, for example, the vehicle 100 may analyze the driver's images captured by the vehicle's exterior cameras to determine that the driver 104 has exited the vehicle 100, and use the signals obtained from the tachograph 102 to determine that the card 106 is still inserted into the tachograph 102. Responsive to such determination, the vehicle 100 may output an alert notification (e.g., via the vehicle's sound exciters or the driver's user device) for the driver 104, which may remind the driver 104 to remove the card 106 from the tachograph 102 while exiting the vehicle 100.

In another exemplary aspect, the vehicle 100 may cause the vehicle's interior and exterior cameras to capture images of vehicle's interior portion and surroundings when the driver 104 removes the card 106 from the tachograph 102 (as determine via the signals obtained from the tachograph 102), to document the vehicle's parking location and/or condition. These images may be stored on an external server and/or a vehicle memory (and/or the card 106 when the driver 104 re-inserts the card 106 later on), so that the authorities and/or the fleet manager can retrieve and access them to check whether the driver 104 is adhering to the defined best practices. In some aspects, during such instances, the vehicle 100 may additionally record and store information associated with the vehicle's radar distances from adjacent vehicles, the tire's inflation values, and/or the like, which may facilitate the authorities and/or the fleet manager to holistically evaluate the driver's performance.

In another exemplary aspect, the vehicle 100 may output an alert notification when the vehicle 100 determines that the driver's drive time is close to exceeding the allocated drive time left on the card 106. In this case, as an example, the vehicle 100 may determine the driver's actual drive time based on the inputs obtained from the vehicle's control unit (shown as VCU 208 in FIG. 2) and compare it with the allocated drive time that is recorded/stored on the card 106. The vehicle 100 may output the alert notification when a difference between the actual drive time and the allocated drive time reduces below a predefined threshold. This alert notification may remind the driver 104 about taking rest, and may hence promote healthy driving habits.

In further aspects, the vehicle 100 may output information associated with the rest or break time left for the driver 104 as determined from the card 106 (when the driver 104 may be taking a break) on the driver's user device, so that the driver 104 may accordingly plan the remaining rest time. The vehicle 100 may further output daily/weekly drive and break time reports on the user devices associated with the driver 104 and the fleet manager. In addition, the vehicle 100 may enable the fleet manager to remotely access the driver information associated with the driver 104, which may be stored on an external server. This may eliminate the need for the driver 104 to access such information by taking print out from the tachograph 102.

The vehicle 100 may further use the inputs obtained from the vehicle's sensor unit to check if the driver's respiration or heartbeat is increased (e.g., has become greater than a predefined healthy/health threshold), and may output a recommendation for the driver 104 to take rest or increase the rest time responsive to such determination. In this case, as an example, the vehicle 100 may obtain inputs or driver images captured by the vehicle's radar sensors or thermal image sensors, and compare the captured images with pre-stored driver images (e.g., driver images when the driver 104 may be healthy). If the captured images are substantially different from the pre-stored driver images, the vehicle 100 may determine that the driver's respiration or heartbeat may have become greater than the predefined health threshold. Responsive to such determination, the vehicle 100 may output the recommendation described above.

The vehicle 100 may further monitor a vehicle battery's state of charge (SoC) level, and perform a remedial action if the SoC level is too low to support the tachograph 102 (or provide power to the tachograph 102) during an expected off or break time for the vehicle 100 / driver 104. In this case, as an example, the vehicle 100 may pre-store a typical power draw profile of the tachograph 102, and use it to calculate an expected total power that the tachograph 102 may draw during the vehicle's expected off or break time. The vehicle 100 may then compare the calculated expected total power with the battery's SoC level when the vehicle 100 enters the off or break time. The vehicle 100 may perform the remedial action described above when the battery's SoC level may be lower than the expected total power that the tachograph 102 may draw during the vehicle's off time. In an exemplary aspect, the remedial action may include running a fast idle to charge the vehicle battery or switching/keeping ON the vehicle engine for a predefined time duration to charge the vehicle battery so that it can optimally support the tachograph 102 during the expected off time.

In additional aspects, the vehicle 100 may predict an estimated amount of tacho paper that may be required on a vehicle trip/journey (e.g., based on the trip's distance), and output a notification for the driver 104 to carry the estimated amount of tacho paper on the vehicle 100. This may ensure that the driver 104 may conveniently take a print out of the driver's driving history and show it the authorities, as and when required during the journey.

The vehicle 100 may further use the vehicle's Human-Machine Interface (HMI, which may have a considerably larger display screen than the tachograph's screen) to display useful information associated with the driver 104 (which may be stored on the card 106 or obtained from an external server). For example, the vehicle 100 may cause the HMI to display information associated with the driver's history on the card 106, upcoming week's activities with allowed driving time, countries visited and tolls paid historically, paper left in the tachograph printer, alert notification if the driver 104 is attempting to enter an impermissible input into the tachograph 102, last 15 or 28 days of driving record, crossing border reminder providing details on requirements for country, and/or the like. It may be appreciated that such information, when displayed on a large-screen HMI, may be useful for the driver 104 and may significantly enhance the driver's experience of operating the tachograph 102.

In this manner, the vehicle 100 may provide various additional features and benefits to the driver 104 (and the fleet manager) by integrating the tachograph 102 into the vehicle 100, which are not possible in conventional systems in which the tachograph is added as an aftermarket accessory to the vehicle.

Further vehicle 100 details are described below in conjunction with FIG. 2.

The vehicle 100 implements and/or performs operations, as described here in the present disclosure, in accordance with the owner manual and safety guidelines. In addition, any action taken by the driver 104 based on the notifications provided by the vehicle 100 should comply with all the rules specific to the location and operation of the vehicle 100 (e.g., Federal, state, country, city, etc.). The notifications, as provided by the vehicle 100, should be treated as suggestions and only followed according to any rules specific to the location and operation of the vehicle 100.

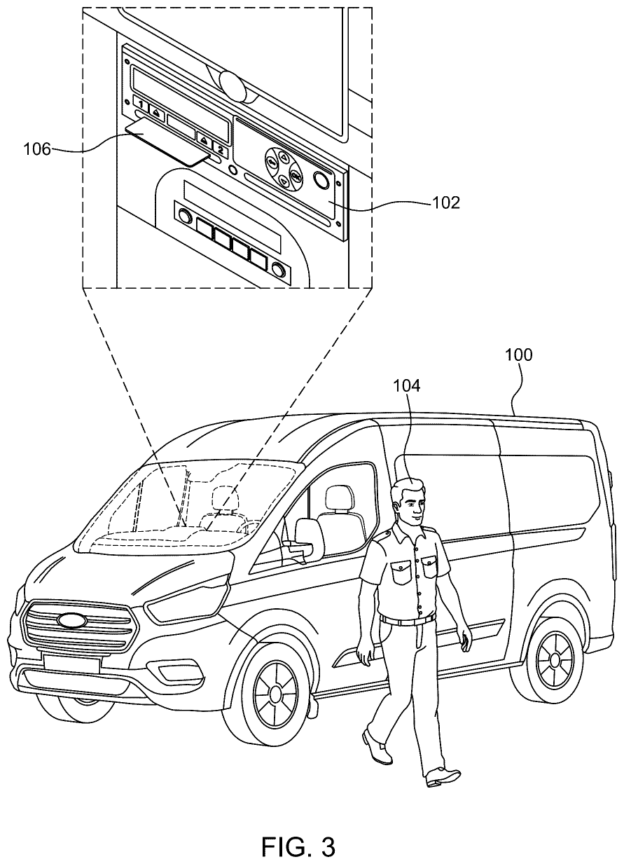

FIG. 2 depicts a block diagram of a system 200 to operate the vehicle 100 with the integrated tachograph 102 in accordance with the present disclosure. While describing FIG. 2, references will be made to FIGS. 3, 4 and 5.

The system 200 may include the vehicle 100, a user device 201 and one or more servers 202 (or a server 202) that may be communicatively coupled with each other via one or more networks 204. The user device 201 may be associated with the driver 104, and may be, for example, a mobile phone, a laptop, a computer, a tablet, a smartwatch, or any other similar device with communication capabilities. The server 202 may be part of a cloud-based computing infrastructure and may be associated with and/or include a Telematics Service Delivery Network (SDN) that provides digital data services to the vehicle 100 and other vehicles (not shown in FIG. 2) that may be part of a vehicle fleet. In further aspects, the server 202 may be associated with the fleet manager that manages the vehicle 100 (and other fleet vehicles). The server 202 may further store historical and upcoming trip information associated with the vehicle 100 and the driver 104. In further aspects, the server 202 may store the driver information associated with the driver 104, which the server 202 may obtain from the vehicle 100 or via the tachograph 102/card 106.

The network(s) 204 illustrates an example communication infrastructure in which the connected devices discussed in various embodiments of this disclosure may communicate. The network(s) 204 may be and/or include the Internet, a private network, public network, or other configuration that operates using any one or more known communication protocols such as transmission control protocol/Internet protocol (TCP/IP), Bluetooth®, Bluetooth Low Energy (BLE), Wi-Fi based on the Institute of Electrical and Electronics Engineers (IEEE) standard 802.11, Ultra-wideband (UWB), and cellular technologies such as Time Division Multiple Access (TDMA), Code Division Multiple Access (CDMA), High-Speed Packet Access (HSPDA), Long-Term Evolution (LTE), Global System for Mobile Communications (GSM), and Fifth Generation (5G), to name a few examples.

The vehicle 100 may include a plurality of units including, but not limited to, an automotive computer 206, a Vehicle Control Unit (VCU) 208 and a tachograph control unit 210 (or unit 210). The VCU 208 may include a plurality of Electronic Control Units (ECUs) 212 in communication with the automotive computer 206.

In some aspects, the automotive computer 206 and/or the unit 210 may be installed anywhere in the vehicle 100, in accordance with the disclosure. Further, the automotive computer 206 may operate as a functional part of the unit 210. The automotive computer 206 may be or include an electronic vehicle controller, having one or more processor(s) 214 and a memory 216. Moreover, the unit 210 may be separate from the automotive computer 206 (as shown in FIG. 2) or may be integrated as part of the automotive computer 206.

The processor(s) 214 may be in communication with one or more memory devices in communication with the respective computing systems (e.g., the memory 216 and/or one or more external databases not shown in FIG. 2). The processor(s) 214 may utilize the memory 216 to store programs in code and/or to store data for performing aspects in accordance with the disclosure. The memory 216 may be a non-transitory computer-readable medium or memory storing a tachograph control program code. The memory 216 may include any one or a combination of volatile memory elements (e.g., dynamic random-access memory (DRAM), synchronous dynamic random-access memory (SDRAM), etc.) and may include any one or more nonvolatile memory elements (e.g., erasable programmable read-only memory (EPROM), flash memory, electronically erasable programmable read-only memory (EEPROM), programmable read-only memory (PROM), etc.).

In accordance with some aspects, the VCU 208 may share a power bus with the automotive computer 206 and may be configured and/or programmed to coordinate the data between vehicle 100 systems, connected servers (e.g., the server(s) 202), the user device 201 and other vehicles (not shown in FIG. 2) operating as part of a vehicle fleet. The VCU 208 may include or communicate with any combination of the ECUs 212, such as a Body Control Module (BCM) 218, an Engine Control Module (ECM) 220, a Transmission Control Module (TCM) 222, a Telematics Control Unit (TCU) 224, a Driver Assistances Technologies (DAT) controller 226, etc.

The VCU 208 may further include and/or communicate with a Vehicle Perception System (VPS) 228, having connectivity with and/or control of one or more vehicle sensory system(s) 230 (or a “sensor unit”). The vehicle sensory system 230 may include one or more vehicle sensors including, but not limited to, a radio detection and ranging (radar) sensor configured for detection and localization of objects inside and outside the vehicle 100 using radio waves, sitting area buckle sensors, sitting area sensors, a light detecting and ranging (lidar) sensor, door sensors, proximity sensors, temperature sensors, wheel sensors, ambient weather sensors, ambient light sensors, vehicle interior and exterior cameras, one or more rain sensors, a humidity sensor, a tire pressure sensor, ultrasonic sensors, one or more microphones, etc. The vehicle sensory system 230 may capture inputs associated with the vehicle's interior portion and vehicle's surroundings (e.g., interior and exterior images, driver's audible or gesture commands, and/or the like).

In some aspects, the VCU 208 may control vehicle operational aspects and implement one or more instruction sets received from the user device 201, from one or more instruction sets stored in the memory 216, including instructions operational as part of the unit 210.

The TCU 224 may provide vehicle connectivity to wireless computing systems onboard and off board the vehicle 100 and may include a Navigation (NAV) receiver 232 for receiving and processing a GPS signal, a BLE Module (BLEM) 234, a Wi-Fi transceiver, a UWB transceiver, and/or other wireless transceivers (not shown in FIG. 2) that may be configurable for wireless communication (including cellular communication) between the vehicle 100 and other systems (e.g., the user device 201, a key fob, an NFC device, etc.), computers, and modules. The NAV receiver 232 may be configured to determine a real-time vehicle geolocation. The TCU 224 may be in communication with the ECUs 212 by way of a bus. In some aspects, the V2X antenna that provides location based inputs to the tachograph 102 may be part of the TCU 224.

The ECUs 212 may control aspects of vehicle operation and communication using inputs from human drivers, inputs from an autonomous vehicle controller, the unit 210, and/or via wireless signal inputs received via the wireless connection(s) from other connected devices, such as the user device 201, the server(s) 202, among others.

The BCM 218 generally includes integration of sensors, vehicle performance indicators, and variable reactors associated with vehicle systems and may include processor-based power distribution circuitry that can control functions associated with the vehicle body such as lights, windows, security, camera(s), fan, headlights, audio system(s), speakers, wipers, door locks and access control, mirrors, various comfort controls, and/or the like. The BCM 218 may also operate as a gateway for bus and network interfaces to interact with remote ECUs (not shown in FIG. 2).

The DAT controller 226 may provide Level-1 through Level-3 automated driving and driver assistance functionality that may include, for example, active parking assistance, vehicle backup assistance, and adaptive cruise control, among other features. The DAT controller 226 may also provide aspects of user and environmental inputs usable for user authentication.

In some aspects, the automotive computer 206 may connect with an infotainment system 236 (or a vehicle Human-Machine Interface (HMI) 236). The infotainment system 236 may include a touchscreen interface portion and may include voice recognition features, biometric identification capabilities that can identify users based on facial recognition, voice recognition, fingerprint identification, or other biological identification means. In other aspects, the infotainment system 236 may further receive user instructions/inputs via the touchscreen interface portion and/or display notifications/recommendations, navigation maps, etc. on the touchscreen interface portion.

As described above, the vehicle 100 may further include the tachograph 102, which may be integrated with the vehicle 100 (e.g., integrated during the vehicle's manufacturing or assembly process). The vehicle 100 may additionally include a battery 238 that may provide power to the tachograph 102 (and other vehicle components) to enable its operation.

The computing system architecture of the automotive computer 206, the VCU 208, and/or the unit 210 may omit certain computing modules. It should be readily understood that the computing environment depicted in FIG. 2 is an example of a possible implementation according to the present disclosure, and thus, it should not be considered limiting or exclusive.

In accordance with some aspects, the unit 210 may be integrated with and/or executed as part of the ECUs 212. The unit 210, regardless of whether it is integrated with the automotive computer 206 or the ECUs 212, or whether it operates as an independent computing system in the vehicle 100, may include a transceiver 240, a processor 242, and a computer-readable memory 244.

The transceiver 240 may receive information/inputs from one or more external devices or systems, e.g., the user device 201, the server(s) 202, and/or the like via the network 204. The transceiver 240 may further transmit notifications/data/signals to the external devices or systems. In addition, the transceiver 240 may receive information/inputs from vehicle 100 components such as the infotainment system 236, the tachograph 102, the VCU 208 (e.g., the vehicle sensory system 230), and/or the like. Further, the transceiver 240 may transmit notifications/command signals to the vehicle 100 components such as the VCU 208, the infotainment system 236, the BCM 218, etc.

The processor 242 and the memory 244 may be the same as or similar to the processor 214 and the memory 216, respectively. In some aspects, the processor 242 may utilize the memory 244 to store programs in code and/or to store data for performing aspects in accordance with the disclosure. The memory 244 may be a non-transitory computer-readable medium or memory storing the tachograph control program code.

In operation, the processor 242 may obtain inputs (e.g., the trigger signal described below in conjunction with FIG. 1) from the tachograph 102 and determine that the card 106 may be connected to the tachograph 102 based on the obtained inputs, when the driver 104 inserts the card 106 into the tachograph card reader. Responsive to determining that the card 106 is connected to the tachograph 102, the processor 242 may authenticate the driver 104 based on the inputs obtained from the vehicle sensory system 230 and the driver information (e.g., the driver's image) that may be stored on the card 106. Specifically, in this case, the processor 242 may first obtain the driver's image that may be pre-stored on the card 106. The processor 242 may further obtain the real-time driver's image captured by the vehicle's interior camera, and compare the driver's image obtained from the card 106 with the real-time driver image. The processor 242 may determine that the driver 104 is an authentic user and the card 106 belongs to the driver 104 when the two images described above match. The processor 242 may allow the driver 104 to use the vehicle 100 in this case.

On the other hand, the processor 242 may determine that the driver 104 may not be an authentic/authorized user and the card 106 may not belong to the driver 104 when the two images do not match. In this case, the processor 242 may perform a predefined action when the driver 104 is not authenticated. For example, the processor 242 may transmit, via the transceiver 240, an alert notification (or a “first alert notification”) to the server 202 or a user device associated with the fleet manager, informing the fleet manager that an unauthorized user is attempting to drive the vehicle 100. As another example, the processor 242 may disable the driver 104 from driving the vehicle 100 or moving the vehicle 100 out of park mode when the processor 242 determines that the driver 104 may not be an authentic user.

As described above, by integrating the tachograph 102 into the vehicle 100, the vehicle 100/processor 242 may provide a plurality of additional features to the driver 104, which may not be possible in conventional systems. For example, the processor 242 may enable the card 106 to act as a vehicle key. In this case, the processor 242 may enable the driver 104 to start the vehicle 100 responsive to determining that the card 106 is connected to the tachograph 102. Stated another way, the processor 242 may not enable the driver 104 to start the vehicle 100 unless the card 106 is inserted into the tachograph 102. This may prevent a scenario where the driver 104 inadvertently starts and drives the vehicle 100 without inserting the card 106 into the tachograph 102. In further aspects, the processor 242 may enable the driver 104 to move the vehicle 100 out of park responsive to determining that the card 106 is connected to the tachograph 102. Stated another way, the processor 242 may not enable the vehicle 100 to move out of park till the card 106 is inserted into the tachograph 102. This feature acts as a secondary prevention measure to ensure that the driver 104 does not drive the vehicle 100 without inserting the card 106 into the tachograph 102. In some aspects, the processor 242 may also enable the driver 104 to open or lock the vehicle doors via the card 106 (similar to how Phone-as-a-key works).

Further, as described above, the processor 242 may enable the driver 104 to use the vehicle's microphone or the vehicle sensory system 230) to provide inputs to the tachograph 102 or record activities via audible or gesture commands. In this case, the processor 242 may first obtain the driver's audible message or gesture input via the vehicle sensory system 230 (e.g., via the vehicle's interior cameras or microphone). The processor 242 may then identify the information included in the driver's audible message or the gesture input (i.e., the information that the driver 104 desires to store on the card 106). The information may be, for example, an input indicating that the driver 104 is performing some other work different from driving (e.g., loading, unloading, paperwork, etc.) or taking rests, the driver's available status, print requests, and/or the like. Responsive to identifying this information, the processor 242 may store the information in the memory 244, the server 202 and/or the card 106, so that it can be later retrieved and accessed for analysis by the driver 104, the fleet manager, the authorities, and/or the like.

In further aspects, the processor 242 may ensure that the driver 104 does not leave the card 106 inserted into the tachograph 102 while exiting the vehicle 100 or leaving an immediate area of the vehicle 100. In this case, the processor 242 may first determine that the driver 104 may have exited the vehicle 100 based on the inputs obtained from the vehicle sensory system 230. For example, the processor 242 may obtain images captured from the vehicle's exterior cameras and determine that the driver 104 may have exited the vehicle 100 based on the obtained images. The processor 242 may additionally determine that the driver 104 may have existed the vehicle 100 based on signals obtained from the vehicle's sitting area sensors (that are part of the vehicle sensory system 230). As another example, the processor 242 may determine that the driver 104 has exited the vehicle 100 and is leaving the vehicle 100 when the radar distance (as determined via the signals obtained from the radar sensors) between the driver 104 and the vehicle 100 is gradually increasing. Responsive to such determination, the processor 242 may obtain the inputs or the signals from the tachograph 102 to check if the card 106 is still inserted into or connected to the tachograph 102. Responsive to determining that the driver 104 has exited the vehicle 100 while the card 106 is still connected to the tachograph 102 (as shown in the exemplary view of FIG. 3), the processor 242 may output an alert notification (or a “second alert notification”) via the vehicle's sound exciters and/or the user device 201, reminding the driver 104 to remove and collect the card 106.

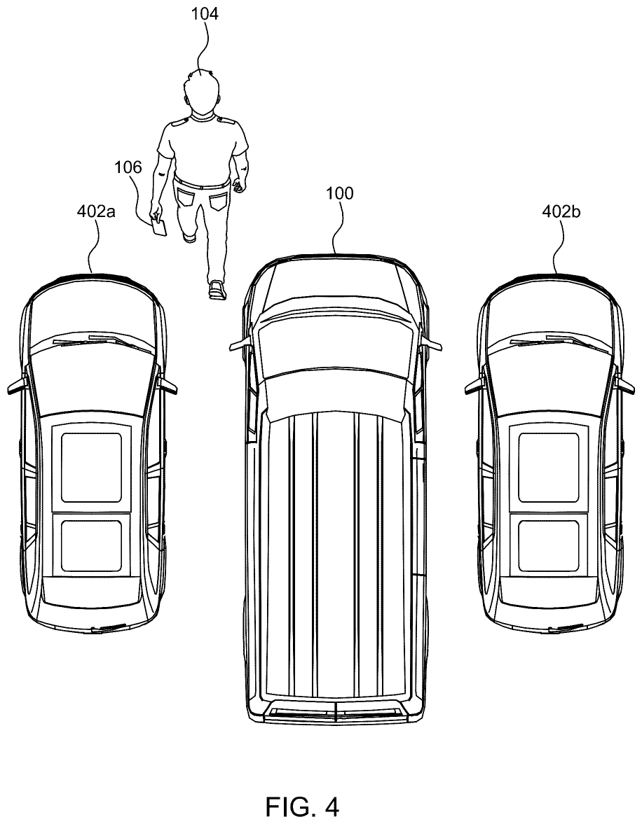

The processor 242 may additionally check if the driver 104 has optimally parked the vehicle 100, when the driver 104 exits the vehicle 100 with the card 106 removed from the tachograph 102. Such parking location/information may be stored in the memory 244, the server 202 and/or the card 106, so that it can be later retrieved and accessed for analysis by the fleet manager and/or the authorities to check the driver's performance. In this case, the processor 242 may first determine that the card 106 is removed from the tachograph 102 based on the inputs/signals obtained from the tachograph 102. Responsive to such determination, the processor 242 may cause the vehicle sensory system 230 to capture images of vehicle's interior portion and surroundings, determine distances from one or more adjacent vehicles 402a, 402b (as shown in FIG. 4) by using the signals obtained from radar, lidar and/or ultrasonic sensors, determine tire inflation values based on inputs obtained from the VCU 208, and/or the like. FIG. 4 depicts an example view of the vehicle 100 parked between the vehicles 402a, 402b, while the driver 104 has exited the vehicle 100 (after removing the card 106 from the tachograph 102).

The processor 242 may then store the captured images, the distances from the adjacent vehicles 402a, 402b, the tire inflation values, and/or the like, in the memory 244, the server 202 and/or the card 106 (when the card 106 is re-inserted into the tachograph 102), so that the fleet manager and/or the authorities may access such information (if required) to check if the driver 104 is optimally parking the vehicle 100. In some aspects, the captured information described above may be time and/or location stamped, so that the fleet manager and/or the authorities may know when and where the vehicle 100 was parked historically.

The processor 242 may further alert the driver 104 if the driver's drive time is closed to exceeding its allocated/maximum limit. In this case, the processor 242 may first determine a maximum drive time associated with the driver 104 based on the driver information stored on the card 106 and/or the driver's schedule information obtained from the server 202. The processor 242 may further obtain the real-time drive time associated with the driver 104 on the inputs obtained from the VCU 208 (e.g., the vehicle sensory system 230). The processor 242 may output an alert notification (or a “third alert notification”) via the HMI 236 and/or the user device 201 when a difference between the maximum drive time and the real-time drive time becomes less than a predefined threshold. Stated another way, the processor 242 may output the alert notification when the driver's drive time is close to exceeding its daily allocated/maximum drive limit.

In further aspects, the processor 242 may determine/extract the information associated with the driver's drive time and break time from the card 106 (e.g., based on the driver information stored on the card 106), and transmit this information to the user device 201. This information may help the driver 104 to know about the driver's remaining rest time when the driver 104 may be taking a break, as described above in conjunction with FIG. 1. The processor 242 may additionally transmit such driver information to the server 202, so that the fleet manager may remotely access this information and check the driver's performance (without having to go to the vehicle 100 and take a print out of the driver information from the tachograph 102).

In additional aspects, the processor 242 may facilitate the driver 104 in driving the vehicle 100 in optimal health condition. In this case, the processor 242 may continuously monitor the driver's respiration or heartbeat based on the inputs obtained from the vehicle sensory system 230 (e.g., images captured by the interior occupancy radar sensors or thermal image sensors). The processor 242 may output (via the HMI 236 and/or the user device 201) a recommendation for the driver 104 to take a break or increase a break time responsive to determining that the driver's respiration or heartbeat has become greater than a predefined health threshold. In this case, as described above in conjunction with FIG. 1, the processor 242 may obtain inputs or driver images captured by the vehicle's radar sensors or thermal image sensors, and compare the captured images with pre-stored driver images (e.g., driver images when the driver 104 may be healthy). If the captured images are substantially different from the pre-stored driver images, the processor 242 may determine that the driver's respiration or heartbeat may have become greater than the predefined health threshold. Responsive to such determination, the processor 242 may output the recommendation described above.

In this manner, the processor 242 may prevent the driver 104 from driving the vehicle 100 when the driver 104 may not be fully fit (or when the driver 104 may not be feeling well).

The processor 242 may further control vehicle operation to ensure that the tachograph 102 is optimally powered by the battery 238. In this case, the processor 242 may first determine an expected driver's rest time or vehicle's off time based on the driver information obtained from the card 106 and/or the driver or vehicle's schedule information obtained from the server 202.

The processor 242 may then estimate an optimal SoC level of the battery 238 that may power the tachograph 102 during the expected vehicle's off time or the driver's rest time, based on the tachograph's typical power draw profile that may be pre-stored in the memory 244. The processor 242 may further determine the real-time battery SoC level closer to the vehicle's off time or the driver's rest time, based on the inputs obtained from the VCU 208. The processor 242 may cause the vehicle engine to be in an ON state or run a fast idle to charge the battery 238 to the optimal SoC level, when the processor 242 determines that the real-time SoC level is less than the optimal SoC level. In this manner, the processor 242 ensures that the tachograph 102 is optimally powered by the battery 238 during the vehicle's off time.

The processor 242 may additionally ensure that the vehicle 100 has sufficient supply of tacho paper for the driver's journey, so that the driver 104 may conveniently take a print out from the tachograph 102, if requested by the authorities. In this case, the processor 242 may first determine an expected trip distance or time duration for the driver's trip based on the driver information obtained from the card 106 or the trip information obtained from the server 202. The processor 242 may then estimate an optimal amount of tacho paper that may be required for the driver's trip based on the expected trip distance or time duration (e.g., by using historical trend information of tacho paper usage on different past trips). In this case, as an example, the processor 242 may correlate the expected trip distance or time duration with the historical trend of tacho paper usage, and determine the optimal amount of tacho paper that may be required for the driver's trip based on the correlation. The processor 242 may further output a notification (or a “fourth notification”) on the HMI 236 and/or the user device 201, indicating the optimal amount of tacho paper. The driver 104 may view/hear the notification and accordingly carry the tacho paper on the trip.

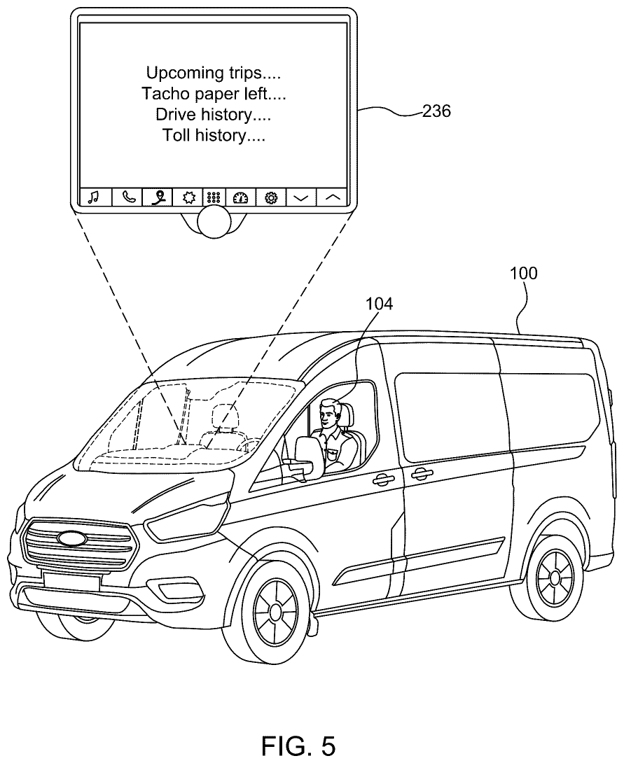

In further aspects, the processor 242 may obtain useful driver and/or trip information from the card 106, the memory 244 and/or the server 202, and display it on the HMI 236 (which may have a larger screen as compared to the screen of the tachograph 102), thereby significantly enhancing the driver's experience of operating the tachograph 102/vehicle 100. An example view of the HMI 236 displaying information for the driver 104 is depicted in FIG. 5.

Examples of such information include, but are not limited to, the driver's history or drive record stored on the card 106, upcoming trips, countries visited in the past by the driver 104 and history of toll paid, tacho paper left in the vehicle 100 or the tachograph 102, crossing border reminder providing details on requirements for the country that the driver 104 may be entering, and/or the like. The processor 242 may further output an alert notification (e.g., a “fifth alert notification”) on the HMI 236 when the processor 242 determines that an input provided by the driver 104 for the tachograph 102 is an impermissible input (e.g., an input that may not be allowed by the fleet manager or may not be in accordance with rules of the country the vehicle 100 is located in). The processor 242 may make such a determination by comparing/correlating the input provided by the driver 104 with information associated with a plurality of impermissible inputs that may be pre-stored in the memory 244. The processor 242 may determine that the driver 104 may be entering an impermissible input when the entered input matches with any one of the plurality of impermissible inputs stored in the memory 244. In this case, the processor 242 may additionally identify and recommend to the driver 104 one or more permissible inputs that the driver 104 may provide to the tachograph 102. For providing this recommendation, the processor 242 may use information associated with a plurality of permissible inputs, which may also be pre-stored in the memory 244.

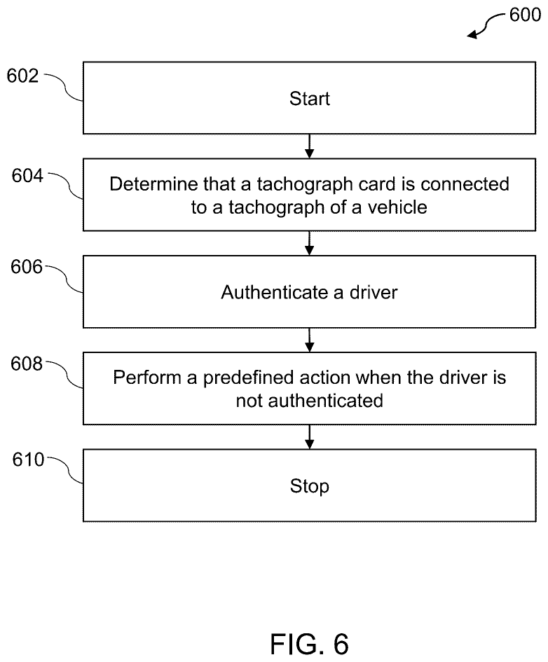

FIG. 6 depicts a flow diagram of an example method 600 to operate the vehicle 100 with the integrated tachograph 102 in accordance with the present disclosure. FIG. 6 may be described with continued reference to prior figures. The following process is exemplary and not confined to the steps described hereafter. Moreover, alternative embodiments may include more or less steps than are shown or described herein and may include these steps in a different order than the order described in the following example embodiments.

The method 600 starts at step 602. At step 604, the method 600 may include determining, by the processor 242, that the card 106 is connected to the tachograph 102 based on the inputs/trigger signal obtained from the tachograph 102. At step 606, the method 600 may include authenticating, by the processor 242, the driver 104 based on the inputs obtained from the vehicle sensory system 230 and the driver information obtained from the card 106, responsive to determining that the card 106 is connected to the tachograph 102.

At step 608, the method 600 may include performing, by the processor 242, a predefined action when the driver 104 is not authenticated. For example, as described above, the processor 242 may transmit an alert notification to the server 202 or disable the driver 104 from driving the vehicle 100, responsive to determining that the driver 104 is not authenticated.

At step 610, the method 600 may stop.

In the above disclosure, reference has been made to the accompanying drawings, which form a part hereof, which illustrate specific implementations in which the present disclosure may be practiced. It is understood that other implementations may be utilized, and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a feature, structure, or characteristic is described in connection with an embodiment, one skilled in the art will recognize such feature, structure, or characteristic in connection with other embodiments whether or not explicitly described.

Further, where appropriate, the functions described herein can be performed in one or more of hardware, software, firmware, digital components, or analog components. For example, one or more application specific integrated circuits (ASICs) can be programmed to carry out one or more of the systems and procedures described herein. Certain terms are used throughout the description and claims refer to particular system components. As one skilled in the art will appreciate, components may be referred to by different names. This document does not intend to distinguish between components that differ in name, but not function.

It should also be understood that the word “example” as used herein is intended to be non-exclusionary and non-limiting in nature. More particularly, the word “example” as used herein indicates one among several examples, and it should be understood that no undue emphasis or preference is being directed to the particular example being described.

A computer-readable medium (also referred to as a processor-readable medium) includes any non-transitory (e.g., tangible) medium that participates in providing data (e.g., instructions) that may be read by a computer (e.g., by a processor of a computer). Such a medium may take many forms, including, but not limited to, nonvolatile media and volatile media. Computing devices may include computer-executable instructions, where the instructions may be executable by one or more computing devices such as those listed above and stored on a computer-readable medium.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating various embodiments and should in no way be construed so as to limit the claims.

Accordingly, it is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their ordinary meanings as understood by those knowledgeable in the technologies described herein unless an explicit indication to the contrary is made herein. In particular, use of the singular articles such as “a,” “the,” “said,” etc. should be read to recite one or more of the indicated elements unless a claim recites an explicit limitation to the contrary. Conditional language, such as, among others, “can,” “could,” “might,” or “may,” unless specifically stated otherwise, or otherwise understood within the context as used, is generally intended to convey that certain embodiments could include, while other embodiments may not include, certain features, elements, and/or steps. Thus, such conditional language is not generally intended to imply that features, elements, and/or steps are in any way required for one or more embodiments.

1. A vehicle comprising:
a tachograph;
a sensor unit configured to capture inputs associated with at least one of a vehicle interior portion or surroundings of the vehicle; and
a processor configured to:
determine that a tachograph card is connected to the tachograph and enable the driver to move the vehicle out of park responsive to determining that the tachograph card is connected to the tachograph;
authenticate a driver based on inputs obtained from the sensor unit and driver information obtained from the tachograph card, responsive to determining that the tachograph card is connected to the tachograph; and
perform a predefined action when the driver is not authenticated.

2. The vehicle of claim 1, wherein the sensor unit comprises at least one of: an interior camera, an exterior camera, a microphone, a Radio Detection and Ranging (radar) sensor, or a Light Detection and Ranging (lidar) sensor.

3. The vehicle of claim 2, wherein the driver information comprises a driver image, and wherein the processor authenticates the driver by comparing the driver image with a real-time image of the driver captured by at least one of the interior camera or the exterior camera.

4. The vehicle of claim 1, wherein, to perform the predefined action, the processor transmits a first alert notification to a server or a user device, or disables the driver from driving the vehicle.

5. The vehicle of claim 1, wherein the processor is further configured to start the vehicle responsive to determining that the tachograph card is connected to the tachograph.

6. (canceled)

7. The vehicle of claim 1, wherein the processor is further configured to:
obtain a driver's audible message or a gesture input via the sensor unit;
identify an information to be stored based on the driver's audible message or the gesture input; and
store the information in at least one of a vehicle memory, an external server, or the tachograph card.

8. The vehicle of claim 1, wherein the processor is further configured to:
determine that the driver has exited the vehicle based on the inputs obtained from the sensor unit;
determine that the tachograph card is still connected to the tachograph after the driver has exited the vehicle; and
output a second alert notification responsive to determining that the driver has exited the vehicle while the tachograph card is still connected to the tachograph.

9. The vehicle of claim 1, wherein the processor is further configured to:
determine that the tachograph card is removed from the tachograph;
responsive to determining that the tachograph card is removed from the tachograph, cause the sensor unit to capture at least one of: images of vehicle's interior portion and surroundings, distances from one or more adjacent vehicles, or tire inflation values; and
store the images, the distances or the tire inflation values in a vehicle memory, an external server or the tachograph card.

10. The vehicle of claim 1, wherein the processor is further configured to:
determine a maximum drive time associated with the driver based on the driver information;
determine a real-time drive time associated with the driver based on the inputs obtained from the sensor unit; and
output a third alert notification when a difference between the maximum drive time and the real-time drive time becomes less than a predefined threshold.

11. The vehicle of claim 1, wherein the processor is further configured to:
determine information associated with driver's drive time and driver's break time based on the driver information obtained from the tachograph card; and
transmit the information associated with driver's drive time and driver's break time to a user device associated with the driver.

12. The vehicle of claim 1, wherein the processor is further configured to:
determine that at least one of a driver's respiration or heartbeat has become greater than a predefined health threshold based on the inputs obtained from the sensor unit; and
output a recommendation for the driver to take a break or increase a break time responsive to determining that the driver's respiration or heartbeat has become greater than the predefined health threshold.

13. The vehicle of claim 1, wherein the processor is further configured to:
determine an expected driver's rest time based on the driver information;
determine a state of charge (SoC) level of a vehicle battery;
determine that the SoC level is less than an optimal SoC level required to supply power to the tachograph during the expected driver's rest time; and
cause a vehicle engine to be in an ON state to charge the vehicle battery responsive to determining that the SoC level is less than the optimal SoC level.

14. The vehicle of claim 1, wherein the processor is further configured to:
determine an expected trip distance or time duration for a driver's trip based on the driver information or a trip information obtained from an external server;
determine an optimal amount of tacho paper required for the driver's trip based on the expected trip distance or time duration; and
output a fourth notification comprising information associated with the optimal amount of tacho paper.

15. The vehicle of claim 1 further comprising a Human-Machine Interface (HMI), wherein the processor is further configured to display the driver information or trip information on the HMI.

16. The vehicle of claim 15, wherein the driver information or trip information comprises information associated with at least one of: a driver's history or drive record stored on the tachograph card, upcoming trips, countries visited in past by the driver and history of toll paid, tacho paper left in the vehicle or the tachograph, or crossing border reminder providing details on requirements for country.

17. The vehicle of claim 15, wherein the processor is further configured to:
determine that an input provided by the driver for the tachograph is an impermissible input; and
output a fifth alert notification via the HMI responsive to determining that the input provided by the driver is impermissible.

18-20. (canceled)

21. A vehicle comprising:
a tachograph;
a sensor unit configured to capture inputs associated with at least one of a vehicle interior portion or surroundings of the vehicle; and
a processor configured to:
determine that a tachograph card is connected to the tachograph;
authenticate a driver based on inputs obtained from the sensor unit and driver information obtained from the tachograph card, responsive to determining that the tachograph card is connected to the tachograph;
perform a predefined action when the driver is not authenticated;
determine that the driver has exited the vehicle based on the inputs obtained from the sensor unit;
determine that the tachograph card is still connected to the tachograph after the driver has exited the vehicle; and
output a second alert notification responsive to determining that the driver has exited the vehicle while the tachograph card is still connected to the tachograph.

22. A vehicle comprising:
a tachograph;
a sensor unit configured to capture inputs associated with at least one of a vehicle interior portion or surroundings of the vehicle; and
a processor configured to:
determine that a tachograph card is connected to the tachograph;
authenticate a driver based on inputs obtained from the sensor unit and driver information obtained from the tachograph card, responsive to determining that the tachograph card is connected to the tachograph;
perform a predefined action when the driver is not authenticated;
determine a maximum drive time associated with the driver based on the driver information;
determine a real-time drive time associated with the driver based on the inputs obtained from the sensor unit; and
output a third alert notification when a difference between the maximum drive time and the real-time drive time becomes less than a predefined threshold.

23. A vehicle comprising:
a tachograph;
a sensor unit configured to capture inputs associated with at least one of a vehicle interior portion or surroundings of the vehicle; and
a processor configured to:
determine that a tachograph card is connected to the tachograph;
authenticate a driver based on inputs obtained from the sensor unit and driver information obtained from the tachograph card, responsive to determining that the tachograph card is connected to the tachograph;
perform a predefined action when the driver is not authenticated;
determine an expected driver's rest time based on the driver information;
determine a state of charge (SoC) level of a vehicle battery;
determine that the SoC level is less than an optimal SoC level required to supply power to the tachograph during the expected driver's rest time; and
cause a vehicle engine to be in an ON state to charge the vehicle battery responsive to determining that the SoC level is less than the optimal SoC level.

24. A vehicle comprising:
a tachograph;
a sensor unit configured to capture inputs associated with at least one of a vehicle interior portion or surroundings of the vehicle; and
a processor configured to:
determine that a tachograph card is connected to the tachograph;
authenticate a driver based on inputs obtained from the sensor unit and driver information obtained from the tachograph card, responsive to determining that the
determine an expected trip distance or time duration for a driver's trip based on the driver information or a trip information obtained from an external server;
determine an optimal amount of tacho paper required for the driver's trip based on the expected trip distance or time duration; and
output a fourth notification comprising information associated with the optimal amount of tacho paper.

25. A vehicle comprising:
a tachograph;
a sensor unit configured to capture inputs associated with at least one of a vehicle interior portion or surroundings of the vehicle; and
a processor configured to:
determine that a tachograph card is connected to the tachograph;
authenticate a driver based on inputs obtained from the sensor unit and driver information obtained from the tachograph card, responsive to determining that the tachograph card is connected to the tachograph; and
perform a predefined action when the driver is not authenticated;
a Human-Machine Interface (HMI), wherein the processor is further configured to display the driver information or trip information on the HMI, wherein the processor is further
determine that an input provided by the driver for the tachograph is an impermissible input; and
output a fifth alert notification via the HMI responsive to determining that the input provided by the driver is impermissible.
